(19) United States
(12) Patent Application Publication
(10) Pub. No.: US 2006/0073577 A1
(43) Pub. Date: Apr. 6, 2006

(54) High succinate producing bacteria

(76) Inventors: San Ka-Yiu, Houston, TX (US); George N. Bennett, Houston, TX (US); Lin Henry, Houston, TX (US); Ailen Sanchez, Houston, TX (US)

Correspondence Address: BAKER & MCKENZIE LLP, Pennzoil Place, South Tower, 711 Louisiana, Suite 3400, HOUSTON, TX, 77002-2716, US

(21) Appl. No.: 11/228,830

(22) Filed: Sep. 16, 2005

Related U.S. Application Data
(60) Provisional application No. 60/610,750, filed on Sep. 17, 2004.

(51) Int. Cl. C12P 13/04; C12P 7/40; C12N 1/21

(52) U.S. Cl. 435/106; 435/136; 435/252.3

(57) ABSTRACT

The invention relates to a hybrid succinate production system that has a high capacity to produce succinate under aerobic and anaerobic conditions. The metabolic engineering of a hybrid bacterial succinate production system that can function under both aerobic and anaerobic conditions makes the production process more efficient, and the process control and optimization less difficult.

### PRIOR RELATED APPLICATIONS

This application claims the benefit of U.S. Provisional Application Serial No. 60/610,750 filed Sep. 17, 2004, entitled “High Succinate Producing Bacteria,” which is incorporated herein in its entirety.

### FEDERALLY SPONSORED RESEARCH STATEMENT

The present invention has been developed with funds from the National Science Foundation. Therefore, the United States Government may have certain rights in the invention.

### REFERENCE TO MICROFICHE APPENDIX

Not applicable.

1. Field of the Invention

The invention relates to a hybrid succinate production system designed in Escherichia coli and engineered to produce a high level of succinate under both aerobic and anaerobic conditions.

2. Background of the Invention

The valuable specialty chemical succinate and its derivatives have extensive industrial applications. Succinic acid is used as a raw material for food, medicine, plastics, cosmetics, and textiles, as well as in plating and waste-gas scrubbing (61). Succinic acid can serve as a feedstock for such plastic precursors as 1,4-butanediol (BDO), tetrahydrofuran, and gamma-butyrolactone. Further, succinic acid and BDO can be used as monomers for polyesters. If the cost of succinate can be reduced, it will become more useful as an intermediary feedstock for producing other bulk chemicals (47). Along with succinic acid, other 4-carbon dicarboxylic acids such as malic acid and fumaric acid also have feedstock potential.

The production of succinate, malate, and fumarate from glucose, xylose, sorbitol, and other “green” renewable feedstocks (in this case through fermentation processes) is an avenue to supplant the more energy intensive methods of deriving such acids from nonrenewable sources. Succinate is an intermediate produced during anaerobic fermentations of propionate-producing bacteria, but those processes result in low yields and concentrations. It has long been known that mixtures of acids are produced from E. coli fermentation. However, for each mole of glucose fermented, only 1.2 moles of formic acid, 0.1-0.2 moles of lactic acid, and 0.3-0.4 moles of succinic acid are produced. As such, efforts to produce carboxylic acids fermentatively have resulted in relatively large amounts of growth substrates, such as glucose, not being converted to desired product.

Succinate is conventionally produced by E. coli under anaerobic conditions. Numerous attempts have been made to metabolically engineer the anaerobic central metabolic pathway of E. coli to increase succinate yield and productivity (7, 8, 12, 14, 15, 20, 24, 32, 44, 48). Genetic engineering coupled with optimization of production conditions have also been shown to increase succinate production. An example is the growth of a succinate producing mutant E. coli strain using dual phase fermentation production mode which comprises an initial aerobic growth phase followed by an anaerobic production phase or/and by changing the headspace conditions of the anaerobic fermentation using carbon dioxide, hydrogen or a mixture of both gases (35, 49). This process is limited by the lack of succinate production during the aerobic phase and the stringent requirement of the anaerobic growth phase for succinate production.

Specifically, manipulating enzyme levels through the amplification, addition, or reduction of a particular pathway can result in high yields of a desired product. Various genetic improvements for succinic acid production under anaerobic conditions have been described that utilize the mixed-acid fermentation pathways of E. coli. One example is the overexpression of phosphoenolpyruvate carboxylase (pepC) from E. coli (34). In another example, the conversion of fumarate to succinate was improved by overexpressing native fumarate reductase (frd) in E. coli (17, 53). Certain enzymes are not indigenous in E. coli, but can potentially help increase succinate production. By introducing pyruvate carboxylase (pyc) from Rhizobium etli into E. coli, succinate production was enhanced (14, 15, 16). Other metabolic engineering strategies include inactivating competing pathways of succinate. When malic enzyme was overexpressed in a host with inactivated pyruvate formate lyase (pfl) and lactate dehydrogenase (ldh) genes, succinate became the major fermentation product (44, 20). An inactive glucose phosphotransferase system (ptsG) in the same mutant strain pfl- and ldh-) had also been shown to yield higher succinate production in E. coli and improve growth (8).

Metabolic engineering has the potential to considerably improve process productivity by manipulating the throughput of metabolic pathways. Specifically, manipulating enzyme levels through the amplification, addition, or deletion of a particular pathway can result in high yields of a desired product. A hybrid succinate production system allows succinate production under both aerobic and anaerobic conditions. Uncoupling succinate production from the oxygen state of the environment has the potential to allow large quantities of succinate to be produced.

The steps involved explain two optimal pathway designs that were first generated from mathematical modeling of the aerobic and anaerobic central pathways of a bacterial species. Proteins can be inactivated as dictated by the optimal design of both conditions. Addition of proteins essential to improving carboxylic acid production can also be activated or overexpressed.

### SUMMARY OF THE INVENTION

Bacteria with a hybrid carboxylic acid production system designed to function under both aerobic and anaerobic conditions are described. The bacteria have inactivated proteins which increase the production of succinate, fumarate, malate, oxaloacetate, or glyoxylate continuously under both aerobic and anaerobic conditions. Inactivated proteins can be selected from ACEB, ACKA, ADHE, ARCA, FUM, ICLR, MDH, LDHA, POXB, PTA, PTSG, and SDHAB. In one embodiment of the invention ACKA, ADHE, ICLR, LDHA, POXB, PTA, PTSG and SDHAB are inactivated. In another embodiment of the invention various combinations of ACEB, ACKA, ADHE, ARCA, FUM, ICLR, MDH, LDHA, POXB, PTA, PTSG, and SDHAB are inactivated to engineer production of a carboxylic acid selected from succinate, fumarate, malate, oxaloacetate, and glyoxylate. Inactivation of these proteins can be combined with overexpression of ACEA, ACEB, ACEK, ACS, CITZ, FRD, GALP, PEPC, and PYC to further increase succinate yield.

In one embodiment of the invention, disruption strains are created wherein the ackA, adhE, arcA, fum, iclR, mdh, ldhA, poxB, pta, ptsG, and sdhAB genes are disrupted. In another embodiment of the invention various combinations of ackA, adhE, arca, fum, iclR, mdh, ldhA, poxB, pta, ptsG, and sdhAB are disrupted. Mutant strains whose genotypes comprise Δ(ackA-pta)-sdhAB-poxB-iclR-ptsG-ldhA-adhE, Δ(ackA-pta)-fum-poxB-iclR-ptsG-ldhA-adhE, Δ(ackA-pta)-mdh-poxB-iclR-ptsG-ldhA -adhE, Δ(ackA-pta)-sdhAB-poxB-ptsG-ldhA-adhE, Δ(ackA-pta)-sdhAB-poxB-iclR-ldhA-adhE, and Δ(ackA-pta)-sdhAB-poxB-ldhA-adhE are described. The strains SBS552MG (ΔadhE ldhA poxB sdh iclR Δack-pta::Cm^{R}, Km^{S}); MBS553MG (ΔadhE ldhA poxB sdh iclR ptsG Δack-pta::Cm^{R}, Km^{S}); and MBS554MG (ΔadhE ldhA poxB sdh iclR ptsG galP Δack-pta::Cm^{R}, Km^{S}) provide non-limiting examples of the succinate production strains. These strains are also described wherein ACEA, ACEB, ACEK, FRD, PEPC, and PYC are overexpressed to further increase succinate yield.

Further, aerobic, anaerobic, and aerobic/anaerobic methods of producing carboxylic acids with a mutant bacterial strain are described, by inoculating a culture with a mutant bacterial strain described above, culturing the bacterial strain under aerobic conditions, culturing the bacterial under anaerobic conditions, and isolating carboxylic acids from the media. Bacteria strains can be cultured in a flask, a bioreactor, a chemostat bioreactor, or a fed batch bioreactor to obtain carboxylic acids. In one example, carboxylic acid yield is further increased by culturing the cells under aerobic conditions to rapidly achieve high levels of biomass and then continuing to produce succinate under anaerobic conditions to increase succinate yield.

Bacterial strains and methods of culture are described wherein at least 2 moles of carboxylic acid are produced per mole substrate, preferably at least 3 moles of carboxylic acid are produced per mole substrate.

### BRIEF DESCRIPTION OF THE DRAWINGS

FIG. 1 Design and Construction of a Hybrid Succinate Production System.

FIG. 2 Hybrid Succinate Production System in E. coli.

### DESCRIPTION OF EMBODIMENTS OF THE INVENTION

Carboxylic acids described herein can be a salt, acid, base, or derivative depending on structure, pH, and ions present. For example, the terms “succinate” and “succinic acid” are used interchangeably herein. Succinic acid is also called butanedioic acid (C₄H₆O₄). Chemicals used herein include formate, glyoxylate, lactate, malate, oxaloacetate (OAA), phosphoenolpyruvate (PEP), and pyruvate. Bacterial metabolic pathways including the Krebs cycle (also called citric acid, tricarboxylic acid, or TCA cycle) can be found in Principles of Biochemistry, by Lehninger as well as other biochemistry texts.

The terms “operably associated” or “operably linked,” as used herein, refer to functionally coupled nucleic acid sequences.

“Reduced activity” or “inactivation” is defined herein to be at least a 75% reduction in protein activity, as compared with an appropriate control species. Preferably, at least 80, 85, 90 , 95% reduction in activity is attained, and in the most preferred embodiment, the activity is eliminated (100%). Proteins can be inactivated with inhibitors, by mutation, or by suppression of expression or translation, and the like.

“Overexpression” or “overexpressed” is defined herein to be at least 150% of protein activity as compared with an appropriate control species. Overexpression can be achieved by mutating the protein to produce a more active form or a form that is resistant to inhibition, by removing inhibitors, or adding activators, and the like. Overexpression can also be achieved by removing repressors, adding multiple copies of the gene to the cell, or up-regulating the endogenous gene, and the like.

The terms “disruption” and “disruption strains,” as used herein, refer to cell strains in which the native gene or promoter is mutated, deleted, interrupted, or down regulated in such a way as to decrease the activity of the gene. A gene can be completely (100%) reduced by knockout or removal of the entire genomic DNA sequence. Use of a frame shift mutation, early stop codon, point mutations of critical residues, or deletions or insertions, and the like, can completely inactivate (100%) gene product by completely preventing transcription and/or translation of active protein.

As used herein “recombinant” is relating to, derived from, or containing genetically engineered material.

Genes are abbreviated as follows: isocitrate lyase (aceA a.k.a. icl); malate synthase (aceB); the glyoxylate shunt operon (aceBAK); isocitrate dehydrogenase kinase/phosphorylase (aceK); acetate kinase-phosphotransacetylase (ackA-pta); aconitate hydratase 1 and 2 (acnA and acnB); acetyl-CoA synthetase (acs); alcohol dehydrogenase (adhE); aerobic respiratory control regulator A and B (arcAB); peroxide sensitivity (arg-lac); alcohol acetyltransferases 1 and 2 (atf1 and atf2); putative cadaverine/lysine antiporter (cadR); citrate synthase (citZ); fatty acid degradation regulon (fadR); fumarate reductase (frd); fructose regulon (fruR); fumarase A, B, or C (fumABC); galactose permease (gaiP); isocitrate dehydrogenase (icd); isocitrate lyase (icl); aceBAK operon repressor (iclR); lactate dehydrogenase (ldhA); malate dehydrogenase (mdh); phosphoenol pyruvate carboxylase (pepC); pyruvate formate lyase (pfl); pyruvate oxidase (poxB); phosphotransferase system genes F and G (ptsF and ptsG); pyruvate carboxylase (pyc); guanosine 3′, 5′-bispyrophosphate synthetase I (relAI); ribosomal protein S12 (rpsL); and succinate dehydrogenase (sdh). Δlac(arg-lac)205(U169) is a chromosomal deletion of the arg-lac region that carries a gene or genes that sensitizes cells to H₂O₂ (51). PYC can be derived from various species, Lactococcus lactis pyc is expressed as one example (AF068759).

Abbreviations: ampicillin (Ap); oxacillin (Ox); carbenicillin (Cn); chloramphenicol (Cm); kanamycin (Km); streptomycin (Sm); tetracycline (Tc); nalidixic acid (Nal); erythromycin (Em); ampicillin resistance (Ap^{R}); thiamphenicol/chloramphenicol resistance (Thi^{R}/Cm^{R}); macrolide, lincosamide and streptogramin A resistance (MLS^{R}); streptomycin resistance (Sm^{R}); kanamycin resistance (Km^{R}); Gram-negative origin of replication (Co1E1); and Gram-positive origin of replication (OriII). Common restriction enzymes and restriction sites can be found at NEB® (NEW ENGLAND BIOLABS®, www.neb.com) and INVITROGEN® (www.invitrogen.com). ATCC®, AMERICAN TYPE CULTURE COLLECTION™ (www.atcc.org).

Plasmids and strains used in certain embodiments of the invention are set forth in Tables 1 and 2. MG1655 is a F^{—}λ^{—} -spontaneous mutant deficient in F conjugation and as reported by Guyer, et al. (18). Pathway deletions were performed using P1 phage transduction and the one-step inactivation based on λ red recombinase (10). The construction of plasmids and mutant E. coli strains were performed using standard biochemistry techniques referenced herein and described in Sambrook (38) and Ausebel (5).

TABLE 1
Plasmids

| Plasmid | Genotype | Ref |
|---|---|---|
| pTrc99A | Cloning vector Ap^{R} | 1 |
| pDHC29 | Cloning vector Cm^{R} | 37 |
| pDHK29 | Cloning vector Km^{R} | 37 |
| pUC19 | Cloning vector Ap^{R} | 60 |
| pHL413 | L. lactis pyc in pTrc99A, Ap^{R} | 40 |
| pCPYC1 | L. lactis pyc Cm^{R} | 54 |
| pHL531 | NADH insensitive citZ in pDHK29, Km^{R} | 41 |
| pLOI2514 | B. subtilis citZ in pCR2.1-TOPO Km^{R}/Ap^{R} | 46 |

TABLE 2
Strains

| Strain | Genotype | Ref | ATCC# |
|---|---|---|---|
| GJT001 | MC4100(ATC35695) cadR mutant | 45 | |
| | Δlac(arg- | | |
| | lac)U169rpsL150relA1ptsF Sm^{R} | | |
| MG1655 | Wild type (F⁻λ⁻) | 18 | 47076 ™ |
| MG1655 | ΔarcA::Km^{R} | 23 | |
| arcA | | | |

| | | |
|---|---|---|
| SBS552MG | ΔadhE ldhA poxB sdh | This Work |
| | iclR Δack-pta::Cm^{R}, Km^{S} | |
| MBS553MG | ΔadhE ldhA poxB sdh | This Work |
| | iclR ptsG Δack-pta::Cm^{R}, Km^{S} | |
| MBS554MG | ΔadhE ldhA poxB sdh | In progress |
| | iclR ptsG Δack- | |
| | pta::Cm^{R}, Km^{S} + GALP | |

For each experiment the strains are freshly transformed with plasmid if appropriate. A single colony is re-streaked on a plate containing the appropriate antibiotics. A single colony is transferred into a 250 ml shake flask containing 50 ml of LB medium with appropriate antibiotics and grown aerobically at 37° C. with shaking at 250 rpm for 12 hours. Cells are washed twice with LB medium and inoculated at 1% v/v into 2 L shake flasks containing 400 ml each of LB medium with appropriate antibiotic concentration and grown aerobically at 37° C. with shaking at 250 rpm for 12 hours. Appropriate cell biomass (˜1.4 gCDW) is harvested by centrifugation and the supernatant discarded. The cells are resuspended in 60 ml of aerobic or anaerobic LB medium (LB broth medium supplemented with 20 g/L of glucose, 1 g/L of NaHCO3) and inoculated immediately into a reactor at a concentration of approximately 10 OD₆₀₀. NaHCO₃ was added to the culture medium because it promoted cell growth and carboxylic acid production due to its pH-buffering capacity and its ability to supply CO₂. Appropriate antibiotics are added depending on the strain.

### EXAMPLE 1

Inhibition of Lactate, Acetate, and Ethanol

A hybrid bacterial strain that produces carboxylic acids under both aerobic and anaerobic conditions can overcome the anaerobic process constraint of low biomass generation. Biomass can be generated under aerobic conditions in the beginning of the fermentation process. During this phase, carboxylic acids are produced in large quantities by the aerobic metabolic synthesis pathways, saving time and cost. Once high biomass is obtained, the environment can be switched or allowed to convert to anaerobic conditions for additional conversion of carbon sources to carboxylic acids at high yields. Utilizing the redesigned anaerobic succinate fermentative pathways, carboxylic acid yield is expected to increase to much greater than 2 or 3 moles product per mole glucose.

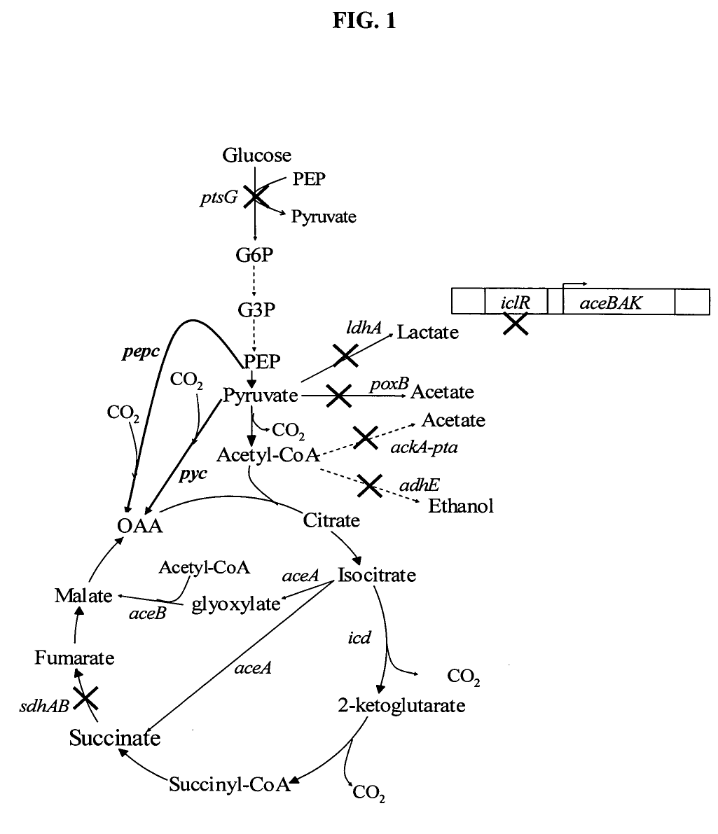

First, to increase flux toward the TCA cycle for carboxylic acid production, two acetate pathways in the aerobic metabolism are inactivated, pyruvate oxidase (POXB) and acetate kinase-phosphotransacetylase (ACKA-PTA) (FIG. 1). Once these two pathways are inactivated, acetate production decreases substantially, and more carbon flux is driven to the TCA cycle.

Additionally, carbon flux through lactate is reduced by inactivating lactate dehydrogenase (LDH). The anaerobic design portion of the hybrid succinate production system consists of multiple pathway inactivations in the mixed-acid fermentation pathways of E. coli. Lactate dehydrogenase (LDHA) and alcohol dehydrogenase (ADHE) are inactivated to conserve both NADH and carbon atoms (FIG. 1). NADH is required in the fermentative carboxylic acid synthesis pathway. Conservation of carbon increases carbon flux toward the fermentative carboxylic acid synthesis pathway.

Next, the glucose phosphotransferase system (PTSG) is also inactivated in order to increase phosphoenolpyruvate (PEP) pool for succinate synthesis (FIG. 1). PEP is a precursor to OAA, which is a major precursor for succinate synthesis. Inactivating PTSG also enhances carbon throughput of the aerobic metabolism. With all these genetic modifications, the aerobic design of the hybrid production system now contains two routes for carboxylic acid production; one is the oxidative branch of the TCA cycle and the other is the glyoxylate cycle.

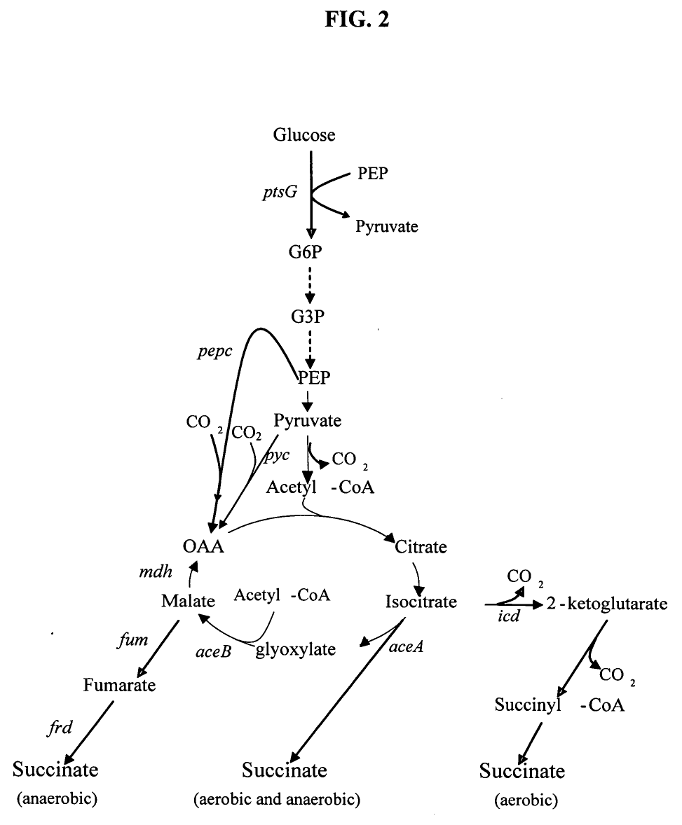

At this point, carboxylic acids are made from the oxidative branch of the TCA cycle. Inactivation of any one of the TCA cycle proteins would create a branched carboxylic acid synthesis pathway. Carbon would flux through both the OAA-malate and citrate-glyoxylate or citrate isocitrate pathways. The branched carboxylic acid pathways, as demonstrated for succinate in FIG. 2, allow continuous production of carboxylic acid product through both aerobic and anaerobic metabolism.

### EXAMPLE 2

Increasing Flux through the Glyoxylate Shunt

As has been previously shown, the presence of native ACEA and ACEB are sufficient to drive carboxylic acid production without requiring additional expression. The native expression level is however susceptible to feedback inhibition and is sensitive the aerobic or anaerobic conditions of the environment. Constitutive activation of the glyoxylate bypass is essential to maintain high levels of aerobic metabolism for carboxylic acid synthesis. This activation is made possible by inactivating the aceBAK operon repressor (ICLR). As seen in FIG. 1, activation of the glyoxylate shunt provides both a mixed fermentive environment which achieves high levels of carboxylic acid production.

### EXAMPLE 3

Succinate Production

Inactivation of succinate dehydrogenase (SDHAB) enables succinate accumulation under aerobic conditions (FIG. 1). Succinate normally does not accumulate under aerobic conditions since it is oxidized in the TCA cycle for supplying electrons to the electron transport chain, and for replenishing oxaloacetate (OAA). The branched metabolic pathway demonstrated in FIG. 2, provides an aerobic, anaerobic and constitutive pathway for carbon flux to generate succinate. The presence of dual-synthesis pathways reduces the need for pure aerobic or pure anaerobic culture conditions allowing industrial culture conditions under lower stringency standards.

### EXAMPLE 4

Carboxylate Production

Succinic acid production is described as a prototypic metabolic pathways for carboxylic acid production. Other carboxylic acids can be produced using this system by inactivating any of the TCA converting enzymes. Notably, the inactivation of fumarase (FUM) will create a branched fumarate production strain. Likewise, inactivation of malate dehydrogenase (MDH) will create a branched malate production strain. Glyoxylate can be produced by inactivating malate synthase (ACEB) and increasing isocitrate dehydrogenase (ACEK) activity.

The production of various bulk specialty chemicals, including fumarate, malate, OAA, and glyoxylate, using bacterial production systems provides a renewable and low cost source for these materials. Using a bacterial strain which produces carboxylic acids under both aerobic and anaerobic conditions reduces constraints on culture conditions thus reducing the cost of bulk chemical production.

### EXAMPLE 5

Increasing Yield

The aerobic and anaerobic network designs for the hybrid succinate production system together include various combinations of gene disruption in E. coli, (ΔsdhAB, ΔackA-pta, ΔpoxB, ΔiclR, ΔptsG, ΔldhA, and ΔadhE). On top of this, pyruvate carboxylase (pyc) and phosphoenolpyruvate carboxylase (pepC) can be co-expressed in the system on a single plasmid (FIG. 1). Increasing PYC and PEPC activity significantly increases the OAA pool for succinate synthesis. PYC converts pyruvate directly to OAA and PEPC converts PEP directly to OAA. Ultimately, the hybrid succinate production contains three routes for succinate synthesis with PYC and PEPC overexpression driving the carbon flux toward these pathways (FIG. 2). The first pathway is the oxidative branch of the TCA cycle, which functions aerobically. The second pathway is the reductive fermentative succinate synthesis pathway, which functions anaerobically. The third pathway is the glyoxylate cycle, which functions aerobically and anaerobically once it is activated.

Further improvements to the hybrid succinate production system include overexpressing malic enzyme to channel pyruvate to the succinate synthesis pathways. This can improve the production rate by reducing any pyruvate accumulation. Pathways in the glyoxylate cycle can also be overexpressed to improve cycling efficiency (i.e. citrate synthase, aconitase, isocitrate lyase, malate synthase). Manipulation of glucose transport systems can also improve carbon throughput to the succinate synthesis pathways. An example is the galactose permease (GALP), which can potentially be used to improve glucose uptake while reducing acetate production. Overexpression of the acetyl-CoA synthetase (ACS) in the presence of externally added acetate is also a potential strategy to further increase the succinate yield. Theoretically, ACS can increase the acetyl-CoA pool at the expense of acetate, while the OAA pool can be just generated from glucose. By decoupling the OAA and acetyl-CoA substrate requirements of the glyoxylate cycle, this can raise the maximum theoretical yield achievable for succinate. Elimination of other pathways that might drain the OAA pool could also enhance the process.

### EXAMPLE 6

Batch Fermentation

As a result of all the strategic genetic manipulations above, a mutant strain of E. coli is created as the hybrid succinate production system (FIG. 2). This mutant strain will be capable of producing high level of succinate no matter what the oxygen tension of the atmosphere is. Certain succinate synthesis routes will always be active to produce succinate independent of the oxygen state of the environment. This factor is very important as it avoids problems with maintaining highly aerated cultures and allows the cells to produce succinate efficiently during the transition from aerobic to anaerobic growth. This ensures a greater flexibility of operation and flexibility in culture protocols. The operational control parameters of the fermenters are greatly widened.

Previously, aerobic batch fermentation was required to increase biomass. Aerobic batch fermentation has been conducted with a medium volume of 600 ml in a 1.0-L NEW BRUNSWICK SCIENTIFIC BIOFLO 110™ fermenter. The temperature was maintained at 37° C., and the agitation speed was constant at 800 rpm. The inlet airflow used was 1.5 L/min. The dissolved oxygen was monitored using a polarographic oxygen electrode (NEW BRUNSWICK SCIENTIFIC™) and was maintained above 80% saturation throughout the experiment. Care was required to maintain aeration and monitor dissolved oxygen concentration. These stringent aerobic growth conditions allow increased biomass at the expense of a large molar carboxylic acid yield. The hybrid carboxylic acid production system reduces oxygen stringency and offers the benefit of an increased biomass and a large product yield.

### EXAMPLE 7

Chemostat Fermentation

Chemostat experiments are performed under aerobic conditions at a dilution rate of 0.1 hr-1. The dilution rate must be customized based on specific growth rates of the bacterial strains, obtained from log phase growth data of previous batch culture studies. A 600 ml batch culture can be maintained chemostatically, using the culture conditions previously described and monitoring the pH using a glass electrode and controlled at 7.0 using 1.5 N HNO₃ and 2 N Na₂CO₃. After inoculation, the culture is allowed to grow in batch mode for 12 to 14 hours before the feed pump and waste pump are turned on to start the chemostat. The continuous culture reached steady state after 5 residence times. Optical density and metabolites are measured from samples at 5 and 6 residence times and then compared to ensure that steady state can be established.

With a hybrid production system, growth conditions can be optimized for carboxylic acid production without stringent boundaries on oxygenation. Subtle changes in culture conditions will not limit the metabolic production, thus oxygenation becomes less critical during the optimization process reducing cost and increasing productivity.

### EXAMPLE 8

Fed Batch Fermentation

Fed batch conducted under aerobic conditions were likewise limited by oxygenation requirements. The initial medium volume is 400 ml in a 1.0-L fermenter as described. Glucose is fed exponentially according to the specific growth rate of the strain studied, obtained from batch experiment results. The program used for glucose feeding is BIOCOMMAND PLUS™ BioProcessing Software from NEW BRUNSWICK SCIENTIFIC™. After inoculation, the culture in the bioreactor is grown in batch mode for up to 14 hrs before the glucose pump is turned on to start the fed batch.

The hybrid carboxylate production system has high capacity to produce bulk carboxylic acids under aerobic and anaerobic conditions. This succinate production system basically can finction under both conditions, which can make the production process more efficient, and the process control and optimization less difficult. Thus, the two steps of most efficient culture growth and production of a large quantity of biomass/biocatalyst can be done under aerobic condition where it is most efficient while succinate is being accumulated, and when oxygen would become limiting at high cell density, the more molar efficient anaerobic conversion process would be dominant. Since there is no need to separate or operationally change the culture during the switch it is easily adaptable to large scale reactors. Carboxylic acid production can be increased to levels much greater than 1 mol carboxylate per mole glucose, some models predict yields as high as 2, 3, or more moles product per mole glucose.

All of the references cited herein are expressly incorporated by reference. References are listed again here for convenience:
1. Alam, et al., J. Bact. 171:6213-7 (1989).
2. Amann, et al., Gene 69:301-15 (1988).
3. Aristodou, et al., Biotechnol. Prog. 11:475-8 (1995).
4. Aristodou, et al. Biotechnol. Bioeng. 63:737-49 (1999).
5. Ausebel, “Current Protocols in Molecular Biology” Greene Pub. Assoc.
6. Berrios-Rivera, et al. Metab. Eng. 4:217-29 (2002).
7. Bunch, et al. Microbiology 143:187-195 (1997).
8. Chatterjee, et al. Appl Environ Microbiol. 67:148-54 (2001).
9. Cox, et al. Development of a metabolic network design and optimization framework incorportating implementation constraints: a succinate production case study. Metab. Eng. Submitted (2005).
10. Datsenko and Wanner. Proc Natl Acad Sci U S A. 97:6640-5 (2000).
11. Dittrich, et al. Biotechnol. Prog. 21:627-31 (2005).
12. Donnelly,et al. App. Biochem. Biotech. 70-72:187-98 (1998).
13. Duckworth and Tong. Biochemistry 15:108-114 (1976).
14. Gokarn, et al., Biotech. Let. 20:795 -8 (1998).
15. Gokarn, et al., Appl. Environ. Microbiol. 66:1844 -50 (2000).
16. Gokam, et al., App. Microbiol. Biotechnol. 56:188-95 (2001).
17. Goldberg, et al., App. Environ. Microbiol. 45:1838-47 (1983).
18. Guyer, et al. Cold Spring Harbor Symp. Quant. Biol. 45:135-40 (1981).
19. Hahm, et al., Appl. Microbiol. Biotechnol. 42:100-7 (1994).
20. Hong and Lee, Appl. Microbiol. Biotechnol. 58:286-90 (2002).
21. Lehninger, et al. “Principles of Biochemistry, 2^{nd} ed.” Worth Pub., New York (1993).
22. Leonard, et al., J. Bact. 175: 870-8 (1993).
23. Levanon, et al. Biotechnol. Bioeng. 89:556-64 (2005).
24. Lin, et al. Biotechnol. Prog. 20:1599-604 (2004).
25. Lin, et al. Biotechnol. Bioeng. 89:148-56 (2005).
26. Lin, H. “Metabolic Network Design and Engineering in E. coli” Ph.D. Thesis, Rice University, Dept. of Bioengineering (2005).
27. Lin, et al. J. Ind. Microbiol. Biotechnol. 32:87-93 (2005).
28. Lin, et al. Metab. Eng. 7:116-27 (2005).
29. Lin, et al. Appl. Microbiol. Biotechnol. 67:515-23 (2005).
30. Lin, et al. Biotechnol. Bioeng. 90:775-9 (2005).
31. Luli and Strohl, Appl. Environ. Microbiol. 56:1004 -11 (1990).
32. Mat-Jan, et al. J. Bact. 171:342-8 (1989).
33. Maurus, et al. Biochemistry 42:5555-65 (2003).
34. Millard, et al.,. App. Environ. Microbiol. 62:1808-10 (1996).
35. Nghiem, et al. U.S. Pat. No. 5,869,301 (1999).
36. Park, et al. J. Bact. 181:2403-10 (1999).
37. Phillips, et al., Biotechniques. 28:400-8 (2000).
38. Sambrook, Fritsch, and Maniatis, “Molecular Cloning—A Laboratory Manual, 2^{nd} ed.” Cold Spring Harbor Laboratory, New York (1989).
39. San, et al. U.S. Application 20050042736.
40. Sanchez, et al. Biotechnol. Prog. 21:358-65 (2005a).
41. Sanchez, et al., Metab. Eng. 7:229-39 (2005b).
42. Sanchez, et al., J. Biotechnol. 117:395-405 (2005c).
43. Stockell et al. J. Biol. Chem. 278:35435-43 (2003).
44. Stols and Donnelly App. Environ. Microbiol. 63:2695-701 (1997).
45. Tolentino et al., Biotech. Let. 14:157-62. (1992).
46. Underwood, et al., App. Environ. Microbiol. 68:1071-81 (2002).
47. Varadarajan and Miller, Biotechnol. Prog. 15:845-54 (1999).
48. Vemuri, et al., Appl. Environ. Microbiol. 68:1715 -27 (2002).
49. Vemuri, et al. J. Ind. Microbiol. Biotechnol. 28:325-32 (2002).
50. Voet and Voet, “Biochemistry 2^{nd} ed.” John Wiley & Sons, New York (1995).
51. Volkert, et al., J. Bact. 176:1297-302 (1994).
52. Wang, et al., J. Biol. Chem. 267:16759-62. (1992).
53. Wang, et al. App. Biochem. Biotechnol. 70-72:919-28 (1998).
54. Wang, et al., App. Environ. Microbiol. 66:1223-7 (2000).
55. Yang, et al. Biotechnol. Bioeng. 65:291-7 (1999).
56. Yang, et al., Metab. Eng. 1:26-34 (1999).
57. Yang, et al., Metab. Eng. 1:141-52 (1999).
58. Yang, et al., Biotechnol. Bioeng. 69:150-9 (2000).
59. Yang, et al., Metab. Eng. 3:115-23 (2001).
60. Yanisch-Perron, et al., Gene 33:103-19 (1985).
61. Zeikus, et al., App. Microbiol. Biotechnol. 51:545-52 (1999).

What is claimed is:

1. A modified bacteria comprising a reduced activity of acetate kinase (ACK), pyruvate oxidase (POXB), and phosphotransacetylase (PTA) proteins, and further comprising reduced activity of one or more proteins selected from the group consisting of alcohol dehydrogenase (ADH), aerobic respiratory control regulator (ARC), fatty acid degradation regulon (FADR), fructose regulon (FRUR), fumarase (FUM), isocitrate dehydrogenase (ICD), isocitrate lyase (ICL), aceBAK operon repressor (ICLR), lactate dehydrogenase (LDH),malate dehydrogenase (MDH), pyruvate formate lyase (PFL), phosphotransferase system F (PTSF), and phosphotransferase system G (PTSG) and succinate dehydrogenase (SDH).

2. The modified bacteria of claim 1, comprising reduced activity of:
a) ACK, ADH, POXB, PTA, and SDH;
b) ACK, ADH, ICLR, LDH, POXB, PTA, and SDH; or
c) ACK, ADH, ICLR, LDH, POXB, PTA, PTSG, and SDH.

3. The modified bacteria of claim 1, comprising reduced activity of ACK, ADH, ICLR, LDH, POXB, PTA, PTSG, and SDH, wherein said bacteria comprise increased activity of GALP.

4. The modified bacteria of claim 1, comprising increased activity of one or more proteins selected from the group consisting of isocitrate lyase (ACEA), malate synthase (ACEB), isocitrate dehydrogenase kinase/phosphorylase (ACEK), aconitase (ACN), acetyl-CoA synthetase (ACS), citrate synthase (CITZ), fumarate reductase (FRD), galactose permease (GALP), phosphoenolpyruvate carboxylase (PEPC), and pyruvate carboxylase (PYC).

5. The modified bacteria of claim 1, comprising a disruption of one or more genes selected from the group consisting of alcohol dehydrogenase (adh), acetate kinase (ack), aerobic respiratory control regulator (arc), fatty acid degradation regulon (fadR), fumarate reductase (frd), fructose regulon (fruR), fumarase (fum), isocitrate dehydrogenase (icd), isocitrate lyase (icl), aceBAK operon repressor (iclR), lactate dehydrogenase (ldh), malate dehydrogenase (mdh), pyruvate formate lyase (pfl), pyruvate oxidase (poxB), phosphotransacetylase (pta), phosphotransferase system F (ptsF), and phosphotransferase system G (ptsG) and succinate dehydrogenase (sdh).

6. The modified bacteria of claim 1, comprising overexpression of one or more genes selected from the group consisting of isocitrate lyase (aceA), malate synthase (aceB); isocitrate dehydrogenase kinase/phosphorylase (aceK), aconitase (acn), acetyl-CoA synthetase (acs), citrate synthase (citZ), galactose permease (galP), phosphoenolpyruvate carboxylase (pepC), and pyruvate carboxylase (pyc).

7. A genetically engineered bacterial cell comprising a disruption of acetate kinase (ack), pyruvate oxidase (poxB), and phosphotransacetylase (pta), and further comprising a disruption of one or more genes selected from the group consisting of alcohol dehydrogenase (adh), aerobic respiratory control regulator (arc), fatty acid degradation regulon (fadR), fumarate reductase (frd), fructose regulon (fruR), fumarase (fum), galactose permease (galP), isocitrate dehydrogenase (icd), isocitrate lyase (icl), aceBAK operon repressor (iclR), lactate dehydrogenase (ldh), malate dehydrogenase (mdh), pyruvate formate lyase (pfI), phosphotransferase system F (ptsF), and phosphotransferase system G (ptsG) and succinate dehydrogenase (sdh).

8. The genetically engineered bacterial cell of claim 7, comprising a disruption of:
a) ack-pta, adh, ldh, poxB, and sdh;
b) ack, adh, iclR, ldh, poxB, pta, and sdh; or
c) ack, adh, iclR, ldh, poxB, pta, ptsG, and sdh.

9. The genetically engineered bacterial cell of claim 7, comprising a disruption of ack, adh, iclR, ldh, poxB, pta, ptsG, and sdh, wherein said bacteria comprise increased activity of GALP.

10. The genetically engineered bacterial cell of claim 7, comprising overexpression of a gene selected from the group consisting of isocitrate lyase (aceA), malate synthase (aceB); isocitrate dehydrogenase kinase/phosphorylase (aceK), aconitase (acn), acetyl-CoA synthetase (acs), citrate synthase (citZ), galactose permease (galP), phosphoenolpyruvate carboxylase (pepC), and pyruvate carboxylase (pyc).

11. A method of producing carboxylic acids in a bacterial culture comprising:
a) providing a bacteria in a culture wherein said bacteria comprises a reduced activity of ACK, POXB, and PTA proteins and further comprising:
i) reduced activity of one or more proteins selected from the group consisting of ADH, ARC, FADR, FRD, FRUR, FUM, ICD, ICL, ICLR, LDH, MDH, PFL, PTA, PTSF, PTSG and SDH,
ii) increased activity of one or more proteins selected from the group consisting of ACEA, ACEB, ACEK, ACN, ACS, CITZ, GALP, PEPC, and PYC, or
iii) both i) and ii);
b) supplying the bacteria with a sugar substrate;
c) allowing the bacteria to metabolize the sugar under aerobic conditions to a sufficient biomass;
d) allowing the bacteria to metabolize the sugar under anaerobic conditions to maximize carboxylic acid production; and
e) recovering the carboxylic acids from the culture.

12. The method of claim 11, wherein said bacteria comprise a disruption of ack, adh, iclR, ldh, poxB, pta, ptsG, and sdh.

13. The method of claim 11, wherein said bacteria comprise a disruption of ack, adh, iclR, ldh, poxB, pta, ptsG, and fum.

14. The method of claim 11, wherein said bacteria comprise a disruption of ack, adh, iclR, ldh, poxB, pta, ptsG, and mdh.

15. The method of claim 11, wherein said bacteria comprise increased activity of a protein selected from the group consisting of isocitrate lyase (ACEA), malate synthase (ACEB), isocitrate dehydrogenase kinase/phosphorylase (ACEK), aconitase (ACN), acetyl-CoA synthetase (ACS), citrate synthase (CITZ), fumarate reductase (FRD), galactose permease (GALP), phosphoenolpyruvate carboxylase (PEPC), and pyruvate carboxylase (PYC).

16. The method of claim 11, wherein said culture is a flask, batch, fed batch, or chemostat culture.

17. The method of claim 11, wherein said culture is a grown under aerobic conditions and carboxylic acid production continues under anaerobic conditions without exchanging head gas.

18. The method of claim 11, wherein said carboxylic acids are recovered at greater than 2 moles carboxylic acid per mol glucose.

19. The method of claim 11, wherein said carboxylic acids are recovered at greater than 3 moles carboxylic acid per mol glucose.
